(19) United States
(12) Patent Application Publication
(10) Pub. No.: US 2025/0050312 A1
(43) Pub. Date: Feb. 13, 2025

(54) PROCESS FOR THE ENANTIOSELECTIVE SEPARATION OF AMINO ACIDS USING CHIRAL POLYSTYRENE MICROSPHERES

(71) Applicant: COUNCIL OF SCIENTIFIC & INDUSTRIAL RESEARCH, New Delhi (IN)

(72) Inventors: Asha SYAMAKUMAR, Pune (IN); Shrikant Babanrao NIKAM, Pune (IN)

(21) Appl. No.: 18/719,595

(22) PCT Filed: Dec. 13, 2022
(86) PCT No.: PCT/IN2022/051071

§ 371 Date: Jun. 13, 2024

(30) Foreign Application Priority Data
Dec. 13, 2021 (IN) 202111058074

(51) Int. Cl. B01J 20/26; B01D 15/38; B01J 20/28; B01J 20/30; C07C 227/40; C08F 8/30

(52) U.S. Cl. CPC B01J 20/265; B01D 15/3833; B01J 20/28019; B01J 20/3028; B01J 20/3071; B01J 20/3085; C07C 227/40; C08F 8/30

(57) ABSTRACT

The present invention relates to an enantioselective separation of racemic mixtures of amino acids like alanine and leucine using chiral polystyrene microspheres. It provides a simple and efficient separation process by using cost-effective commodity polymer like polystyrene after simple post polymer modifications which involves simple filtration without using any high end chromatographic instrument. The present process provides the preparation of chiral modified protected or deprotected polystyrene powder. The present invention provides a process of preparing the modified polystyrene to form the deprotected polystyrene along with the process of preparation of the modified poly styrene compound in fiber form or microspheres form wherein the change in the morphology is observed from irregular to fibrous upon deprotecting the protected polymer. This process achieves high separation efficiency and % Enantiomeric excess values (% ee) value.

### CROSS REFERENCES TO RELATED APPLICATIONS

This application is a national-stage application under 35 U.S.C. § 371 of International Application No. PCT/IN2022/051071, filed Dec. 13, 2022, which International Application claims benefit of priority to Indian Patent Application number 202111058074, filed Dec. 13, 2021.

### TECHNICAL FIELD OF THE INVENTION

The present invention relates to a process for the enantioselective separation of amino acids using chiral polystyrene microspheres. More particularly, the present invention relates to an enantioselective separation of racemic mixtures of alanine and leucine using polystyrene microspheres.

### BACKGROUND OF THE INVENTION

Enantioselective separation of racemic mixtures is an important operation in organic chemistry. Different methods such as chiral chromatography, enantioselective crystallization, and enantioselective filtration are used in the racemic approach to separate enantiomers. Chiral chromatographic methods are most commonly used but are associated with limitations like the high-end instruments and skilled workforce. Methods like enantioselective crystallization need enantiopure sacrificial compounds. Recently, enantioselective filtration methods using chiral selectors have gained more attention from the scientific community since these methods are simple, straightforward, energy-efficient, and do not require high-end instruments. Chiral polymeric microspheres with particle sizes ranging from 1-200 μm with high surface area are increasingly reported as stationary phase in chiral chromatography.

Prior arts for the enantioselective separation by using chiral polymers are available. Such as: In the available solutions, polymers are mostly coated on inorganic materials like modified silica or metal Nanoparticles as chiral selectors. (Angew. Chemie-Int. Ed. 2016, 55 (40), 12460-12464, ACS Appl. Mater. Interfaces 2009, 1 (8), 1834-1842, J. Porous Mater. 2021, 28 (2), 407-414). The microspheres with multiple chiral components were used for carrying out enantioselective separation (Macromol. Chem. Phys. 2020, 221 (24), 1-7, Macromol. Rapid Commun. 2011, 32 (24), 1986-1992.)

Reports are available for using magnetic chiral particles prepared by coating chiral selectors on the metal surfaces where the separation process is tedious and need a magnetic environment to carry out the separation (J. Mater. Chem. B, 2014, 2 (7), 775-782; New J. Chem., 2014, 38 (8), 3630-3636).

Reports are available for using complex chiral MOF to carry out the racemic separation (ACS Appl. Mater. Interfaces 2020, 12 (14), 16903-16911). Different methods like chiral chromatography or enantioselective crystallization are used to carry out separation, which needs high-end instruments, more time or enantiopure sacrificial agent. (Sep. Purif. Technol., 2012, 87, 142-148; Adv. Funct. Mater., 2007, 17 (6), 944-950). All the aforesaid prior art processes are having their own limitations as mentioned above.

Therefore, there is a need in the art to develop a separation process by using commodity polymer like polystyrene after simple post polymer modifications and which involves simple filtration without using any high-end chromatographic instrument.

### OBJECTIVES OF THE INVENTION

The main objective of the present invention is to provide a simple and efficient process for the enantioselective separation of racemic mixtures of amino acids by using polystyrene microspheres.

Yet another objective of the present invention is to provide a process of preparing the chiral modified protected or deprotected polystyrene powder.

### SUMMARY OF THE INVENTION

Accordingly, to accomplish the objectives, the present invention provides a process for the enantioselective separation of amino acids using chiral polystyrene microspheres.

In an aspect, the present invention provides a process for the enantioselective separation of a racemic mixture of native amino acid by using chiral modified polystyrene; wherein the process comprises the steps of:
a) dissolving racemic mixture of native amino acid in water under stirring;
b) adding modified polystyrene into the racemic mixture obtained in step a) to obtain polymer powder suspended racemic mixture;
c) stirring the polymer powder suspended racemic mixture obtained in step b) at a temperature in a range of 25-35° C. for a period in a range of 22-24 hours; and
d) filtering the suspended polymer powder from the mixture obtained at step c) to afford enantioselective separation by getting one enantiomer in filtrate and other adsorbed on the chiral surface of the polymer powder;
wherein the chiral modified polystyrene used in step b) is L-leucine containing polystyrene (PS-L-Leu), which is either protected or deprotected.

In another aspect of the present invention, the amino acid is selected from alanine, leucine, aspartic acid, glutamic acid, lysine, phenylalanine, serine, and valine.

In another aspect of the present invention, the chiral modified polystyrene powder is deprotected polystyrene (PS-L-Leu).

In another aspect of the present invention, the chiral modified polystyrene powder is in a form of fiber or microsphere.

In yet another aspect, the present invention provides a process of preparing the modified polystyrene comprising the steps of:
a) synthesizing the polystyrene by control radical polymerization (CRP) techniques of RAFT polymerization in the temperature range of 110-115° C. for 5-6 hours without using any solvent (bulk polymerization);

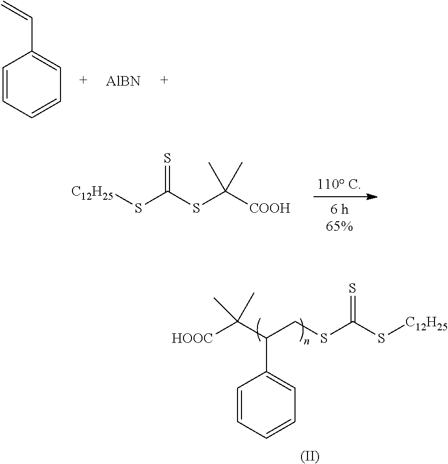

b) reacting the polystyrene obtained in step a) with N-phthaloyl 1-leucine acid chloride in the presence of AlCl₃ at a temperature in the range of 0-5° C. for 1 hour in a 1, 2-dichloroethane as a solvent followed by stirring at 25-35° C. for 2 hours to obtain protected N-Phthaloyl L-leucine containing polystyrene (N-Pth-L-Leu-PS) (protected polystyrene);

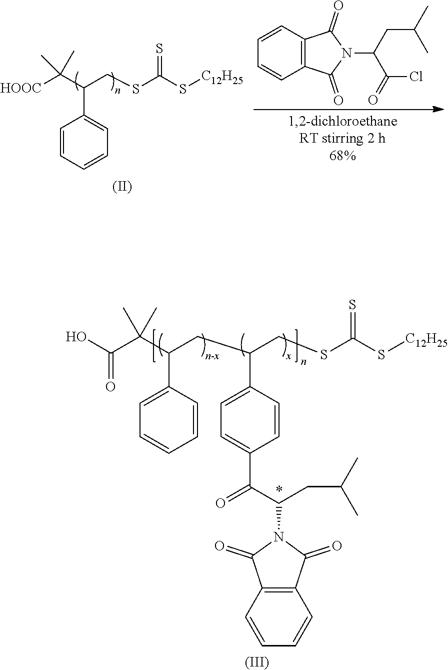

c) deprotecting phthaloyl protection from N-Phthaloyl L-leucine containing polystyrene (N-Pth-L-Leu-PS) obtained at step b) in the presence of hydrazine hydrate at a temperature in the range of 120-125° C. for a period in the range of 22-24 hours in a toluene as a solvent to get L-leucine containing polystyrene (PS-L-Leu) (deprotected polystyrene).

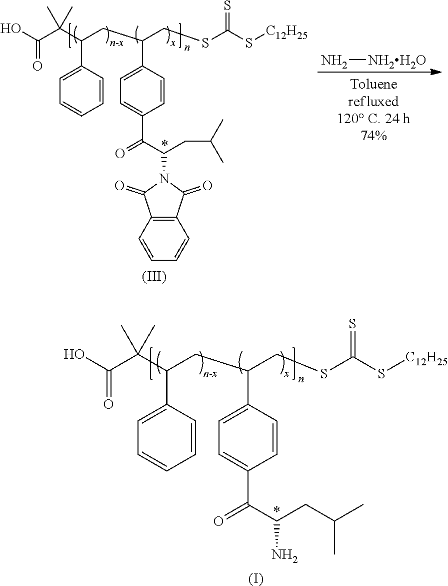

In another aspect of the present invention, the degree of polymerization (n) (in step a) is equal to 218 (calculated from GPC data) (table 2).

In another embodiment of the present invention, in step b) the % modification is calculated taking the ratio of integration of two peaks (7.10-6.58 corresponding to 5H and 5.74-5.70 corresponding to 1 proton) using NMR (FIG. 6) which is found to be 27%.

In another embodiment of the present invention, in step c) the % modification is calculated similarly by taking the ratio of integration of two peaks (7.03-6.47 corresponding to 5H and 4.98-4.93 corresponding to 1 proton) using NMR (FIG. 8) which is found to be 34%.

Yet another aspect, the present invention provides a process for the preparation of modified polystyrene compound in fiber form or microspheres form, wherein said process comprises the steps of:
(a) dissolving modified polystyrene powder (Protected PS-L-Leu or Deprotected PS-L-Leu) in dichloromethane;
(b) adding 4% aqueous polyoxyethylene sorbitan monooleate (tween 80) surfactant in water, surfactant solution into modified polystyrene solution obtained in step a) and homogenizing the reaction mixture at 20000-22000 rpm with homogenizer for 10-15 mins until the solution formed a milky white uniform suspension;
(c) evaporating the dichloromethane from the polymer suspension obtained in step b) by subjecting to prolong stirring at 1500 rpm with an overhead stirrer for a period in the range of 3-4 hours at a temperature in the range of 40-45° C.;
(d) centrifuging the polymer particles suspension mass obtained in step c) at 10000 rpm to settle down the polymeric particles at the bottom and then decanting the supernatant liquid;
(e) washing the settle-down polymer particle obtained at step d) with water and filtering through 0.22 μm pore size PVDF membranes using vacuum filtration assembly; and
(f) drying the particles obtained at step e) in a vacuum oven at a temperature in the range of 40-45° C. for a period in the range of 15-20 hours to afford spherical polymeric particles.

In an embodiment of the present invention, the modified polystyrene powder solution and polyoxyethylene sorbitan monooleate (tween 80) surfactant solution is present in a ratio ranging from 1:3 to 1:5.

In another embodiment of the present invention, the process of centrifuging the polymer particles suspension is repeated 3-4 times by adding fresh water to ensure complete removal of the water-soluble surfactant.

In another embodiment of the present invention, the solvent used is selected from the group consisting of 1, 2-dichloroethane, hydrazine hydrate, toluene, dichloromethane and tween 80 (polyoxyethylene sorbitan monooleate).

In another embodiment of the present invention, the change in morphology is observed from irregular to fibrous upon deprotecting the protected polymer.

### BRIEF DESCRIPTION OF THE DRAWINGS

FIG. 1: CD spectra representing the enantioselective separation of a) alanine b) leucine racemic mixtures by using 5 mg of Protected PS-L-Leu of formula (III) powder/microspheres

FIG. 2: CD spectra representing enantioselective separation of a) alanine b) leucine racemic mixtures by using 5 mg of Deprotected PS-L-Leu of formula (I) fiber/microspheres

FIG. 3: CD spectra representing the enantioselective separation of leucine racemic mixture by using 5, 9 and 12 mg of Deprotected PS-L-Leu of formula (I) microspheres

FIG. 4: ¹H NMR spectrum of Polystyrene formula (II) (CDCl₃)

FIG. 5: ¹³C NMR spectrum of Polystyrene formula (II) (CDCl₃)

FIG. 6: ¹H NMR spectrum of Protected PS-L-Leu of formula (III) (CDCl₃)

FIG. 7: ¹³C NMR spectrum of Protected PS-L-Leu of formula (III) (CDCl₃)

FIG. 8: ¹H NMR spectrum of Deprotected PS-L-Leu of formula (I) (CDCl₃)

FIG. 9: ¹³C NMR spectrum of Deprotected PS-L-Leu of formula (I) (CDCl₃)

FIG. 10: GPC chromatogram of polymers (CHCl₃)

FIG. 11: water contact angles of a) Polystyrene b) Protected PS-L-Leu (III) c) Deprotected PS-L-Leu (I).

FIG. 12: Polymer morphology images (as it is and microspheres) of protected PS-L-Leu (III) and deprotected PS-L-Leu (I) by FE-SEM.

### DETAILED DESCRIPTION OF THE INVENTION

It is to be understood that the figures and descriptions of the present invention have been simplified to illustrate elements that are relevant for a clear understanding of the invention. The detailed description will be provided herein below with reference to the attached drawing.

The present invention provides a process for the enantioselective separation of amino acids using chiral polystyrene microspheres.

In an embodiment, the present invention provides a process for the enantioselective separation of a racemic mixture of native amino acid by using polystyrene microspheres; wherein the process comprises the steps of:
a) dissolving racemic mixture of native amino acid in water under stirring;
b) adding modified polystyrene into the racemic mixture obtained at step a);
c) stirring the polymer powder suspended racemic mixture obtained at step b) at a temperature in the range of 25-35° C. for a period in the range of 22-24 hours;
d) filtering the suspended polymer powder from the mixture obtained at step c) to afford enantioselective separation by getting one enantiomer in filtrate and other adsorbed on the chiral surface of the polymer powder;
wherein, the modified polystyrene used at step b) is L-leucine containing polystyrene (PS-L-Leu), which is either protected or unprotected, fibers or microspheres.

In an embodiment of the present invention, the amino acid at step a) is selected from alanine, leucine, aspartic acid, glutamic acid, lysine, phenylalanine, serine, and valine or mixture thereof. Preferably, the amino acid mixture is alanine and leucine.

In an embodiment of the present invention, a chiral modified polystyrene is represented by formula (I) or formula (III):

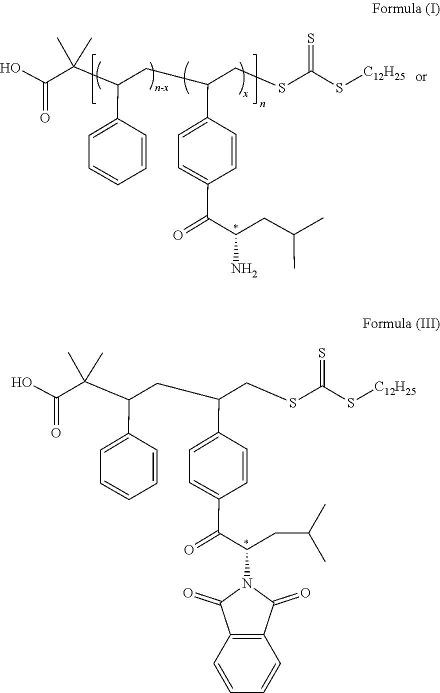

wherein n=200-250; x=68-85; and n−x=132-165.

In another embodiment of the present invention, the chiral modified polystyrene is in a form of powder, fiber or microsphere.

In another embodiment of the present invention, the chiral modified polystyrene is in irregular shape or in fibrous shape.

In another embodiment of the present invention, the chiral modified polystyrene of formula I has 53.4° of water contact angle, and the chiral modified polystyrene of formula III has 95.6° of water contact angle.

In an embodiment, the present invention provides a process for the enantioselective separation of a racemic mixture of native amino acid by using chiral modified polystyrene powder; wherein the process comprises the steps of:
a) dissolving racemic mixture of native amino acid in water under stirring;
b) adding modified polystyrene powder into the racemic mixture obtained in step a) to obtain polymer powder suspended racemic mixture;
c) stirring the polymer powder suspended racemic mixture obtained in step b) at a temperature in a range of 25-35° C. for a period in a range of 22-24 hours; and
d) filtering the suspended polymer powder from the mixture obtained at step c) to afford enantioselective separation by getting one enantiomer in filtrate and other adsorbed on the chiral surface of the polymer powder;
wherein the chiral modified polystyrene powder used in step b) is L-leucine containing polystyrene (PS-L-Leu), which is either protected or deprotected, fibers or microspheres.

In another embodiment, the present invention provides a process for the enantioselective separation of a racemic mixture of native amino acid by using modified deprotected polystyrene powder (PS-L-Leu); wherein the process comprises the steps of:
a) dissolving racemic mixture of native amino acid in water under stirring;
b) adding modified deprotected polystyrene powder (PS-L-Leu) into the racemic mixture obtained in step a) to obtain polymer powder suspended racemic mixture;
c) stirring the polymer powder suspended racemic mixture obtained in step b) at a temperature in a range of 25-35° C. for a period in a range of 22-24 hours; and
d) filtering the suspended polymer powder from the mixture obtained in step c) to afford enantioselective separation by getting one enantiomer in filtrate and other adsorbed on the chiral surface of the polymer powder; wherein the deprotected polystyrene powder (PS-L-Leu) is in the form of microsphere.

In another embodiment, the present invention provides a process for the enantioselective separation of a racemic mixture of native amino acid by using modified protected polystyrene powder (PS-L-Leu); wherein the process comprises the steps of:
a) dissolving racemic mixture of native amino acid in water under stirring;
b) adding modified protected polystyrene powder (N-Pth-L-Leu-PS) into the racemic mixture obtained in step a) to obtain polymer powder suspended racemic mixture;
c) stirring the polymer powder suspended racemic mixture obtained in step b) at a temperature in a range of 25-35° C. for a period in a range of 22-24 hours; and
d) filtering the suspended polymer powder from the mixture obtained in step c) to afford enantioselective separation by getting one enantiomer in filtrate and other adsorbed on the chiral surface of the polymer powder; wherein the protected polystyrene powder (N-Pth-L-Leu-PS) is in the form of microsphere.

In another embodiment, the present invention provides a process for preparation of modified polystyrene comprising the steps of:
(a) synthesizing the polystyrene (II) by control radical polymerization (CRP) techniques of RAFT polymerization in the temperature range of 110-115° C. for 5-6 hours without using any solvent (bulk polymerization);

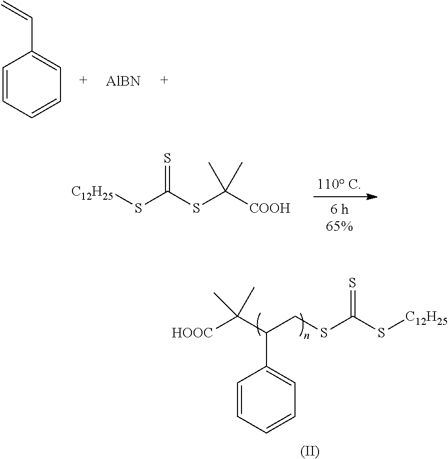

(b) reacting the polystyrene (II) obtained at step i) with N-phthaloyl 1-leucine acid chloride in the presence of AlCl₃ at a temperature in the range of 0-5° C. for 1 hour in a 1, 2-dichloroethane as a solvent followed by stirring at 25-35° C. for 2 hours to obtain protected N-Phthaloyl L-leucine containing polystyrene (N-Pth-L-Leu-PS) of formula (III);

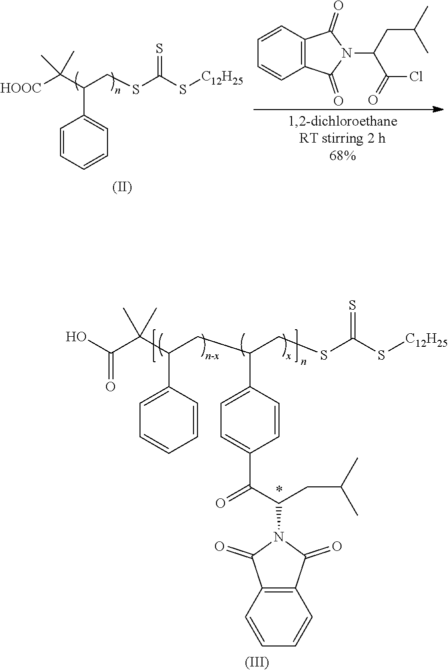

(c) deprotecting N-Phthaloyl L-leucine containing polystyrene (N-Pth-L-Leu-PS) of formula (III) obtained at step ii) in the presence of hydrazine hydrate at a temperature in the range of 120-125° C. for a period in the range of 22-24 hours in a toluene as a solvent to get L-leucine containing polystyrene (PS-L-Leu) of formula (I).

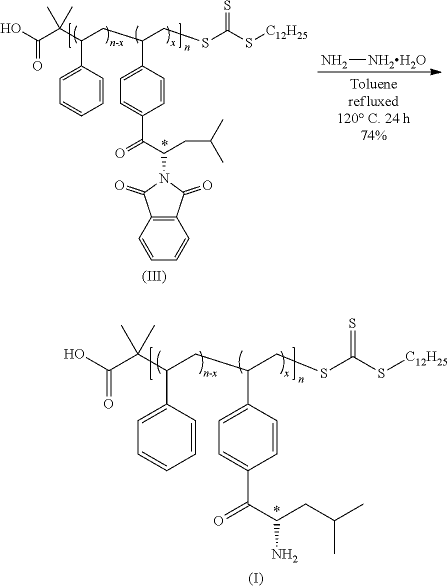

In specific embodiment of the present invention, the degree of polymerization (n) (in step a) is equal to 218 (calculated from GPC data).

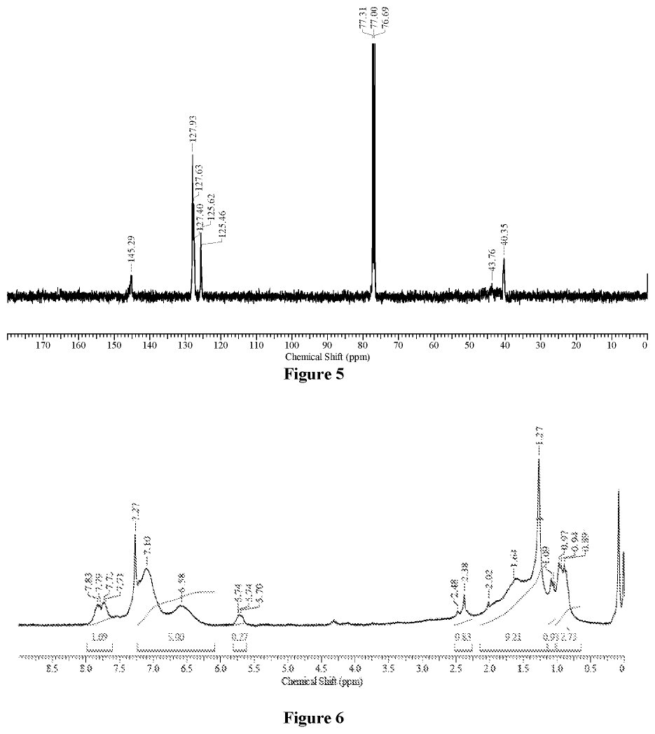

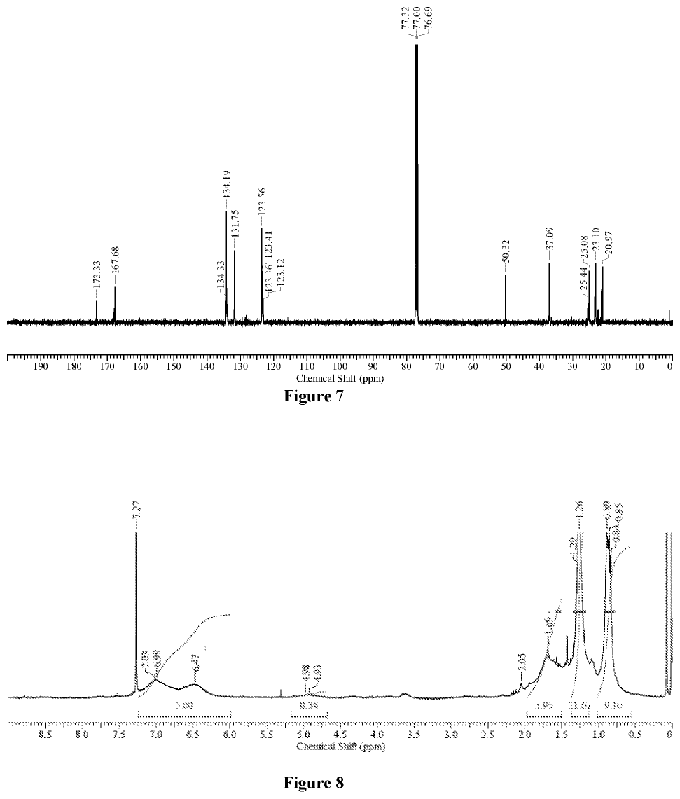

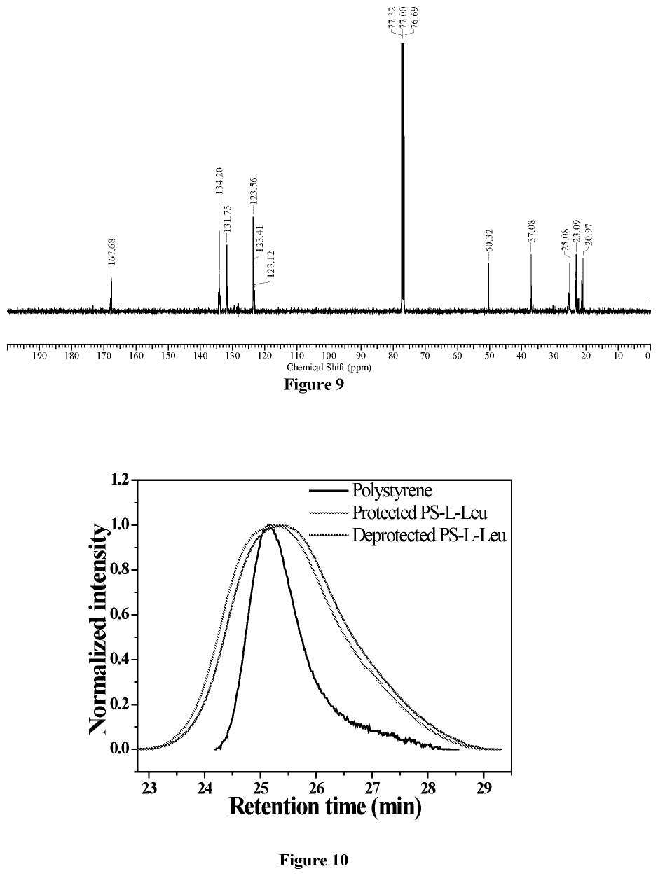

In another embodiment of the present invention, in step b) the % modification is calculated taking the ratio of integration of two peaks (7.10-6.58 corresponding to 5H and 5.74-5.70 corresponding to 1 proton) using NMR (FIG. 6) which is found to be 27%.

In another embodiment of the present invention, in step c) the % modification is calculated similarly by taking the ratio of integration of two peaks (7.03-6.47 corresponding to 5H and 4.98-4.93 corresponding to 1 proton) using NMR (FIG. 8) which is found to be 34%.

According to the present invention, the chirality can be introduced in the polystyrene by different ways like using chiral catalyst, polymerization of chiral styrene monomers or by post polymerization modification. In particularly useful embodiment, post polymerization modification is used for the chirality introduction.

In yet another embodiment, the present invention provides a process for the preparation of modified polystyrene compound in fiber form or microspheres form, wherein said process comprises the steps of:
(a) dissolving modified polystyrene powder (Protected PS-L-Leu or Deprotected PS-L-Leu) in dichloromethane;
(b) adding 4% aqueous tween 80 (polyoxyethylene sorbitan monooleate) surfactant in water, surfactant solution into modified polystyrene solution obtained in step a) and homogenizing the reaction mixture at 20000-22000 rpm with homogenizer for 10-15 mins until the solution formed a milky white uniform suspension;
(c) evaporating the dichloromethane from the polymer suspension obtained in step b) by subjecting to prolong stirring at 1500 rpm with an overhead stirrer for a period in the range of 3-4 hours at a temperature in the range of 40-45° C.;
(d) centrifuging the polymer particles suspension mass obtained in step c) at 10000 rpm to settle down the polymeric particles at the bottom and then decanting the supernatant liquid;
(e) washing the settle-down polymer particle obtained at step d) with water and filtering through 0.22 μm pore size PVDF membranes using vacuum filtration assembly; and
(f) drying the particles obtained at step e) in a vacuum oven at a temperature in the range of 40-45° C. for a period in the range of 15-20 hours to afford spherical polymeric particles.

In another embodiment of the present invention, the solvent used is selected from the group consisting of 1, 2-dichloroethane, toluene, and dichloromethane.

In an embodiment of the present invention, the ratio of modified polystyrene powder solution to tween 80 surfactant solution is in a range of 1:3 to 1:5. Preferably, the ratio is 1:4.

In another embodiment of the present invention, the change in morphology is observed from irregular to fibrous upon deprotecting the protected polymer.

In another embodiment, the present invention provides a process for the enantioselective separation of a racemic mixture of native amino acid; wherein the process comprises the steps of:
(a) synthesizing the polystyrene (II) by control radical polymerization (CRP) techniques of RAFT polymerization in the temperature range of 110-115° C. for 5-6 hours without using any solvent (bulk polymerization);

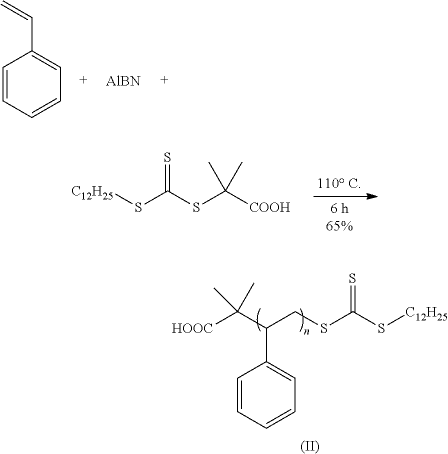

(b) reacting the polystyrene (II) obtained at step i) with N-phthaloyl 1-leucine acid chloride in the presence of AlCl₃ at a temperature in the range of 0-5° C. for 1 hour in a 1, 2-dichloroethane as a solvent followed by stirring at 25-35° C. for 2 hours to obtain protected N-Phthaloyl L-leucine containing polystyrene (N-Pth-L-Leu-PS) of formula (III);

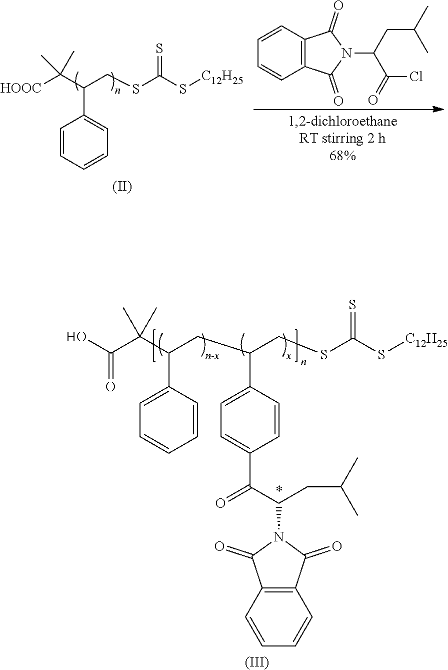

(c) deprotecting N-Phthaloyl L-leucine containing polystyrene (N-Pth-L-Leu-PS) of formula (III) obtained at step ii) in the presence of hydrazine hydrate at a temperature in the range of 120-125° C. for a period in the range of 22-24 hours in a toluene as a solvent to get L-leucine containing polystyrene (PS-L-Leu) of formula (I);

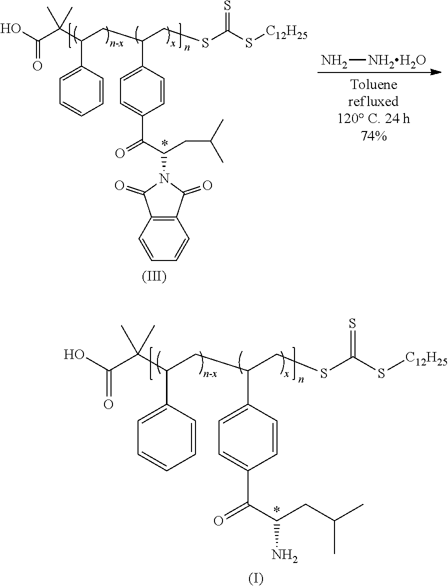

(d) dissolving racemic mixture of native amino acid in water under stirring to obtain amino acid racemic mixture and adding the protected or deprotected polystyrene powder into the amino acid racemic mixture;
(e) stirring the polymer powder suspended racemic mixture obtained at step d) at a temperature in the range of 25-35° C. for a period in the range of 22-24 hours;
(f) filtering the suspended polymer powder from the mixture obtained at step e) to afford enantioselective separation by getting one enantiomer in filtrate and other adsorbed on the chiral surface of the polymer powder.

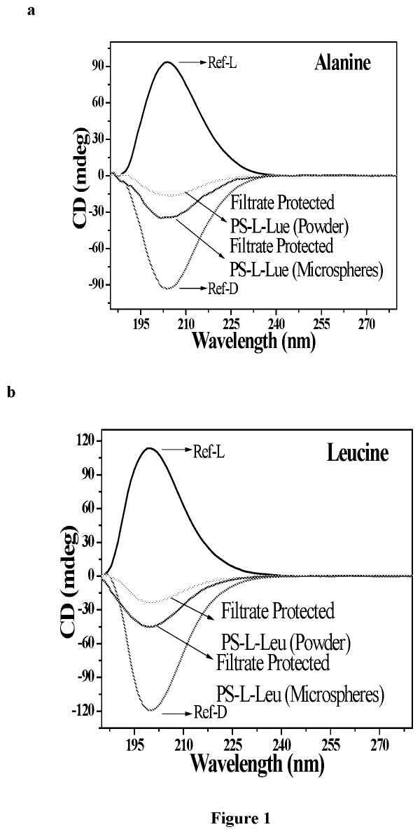

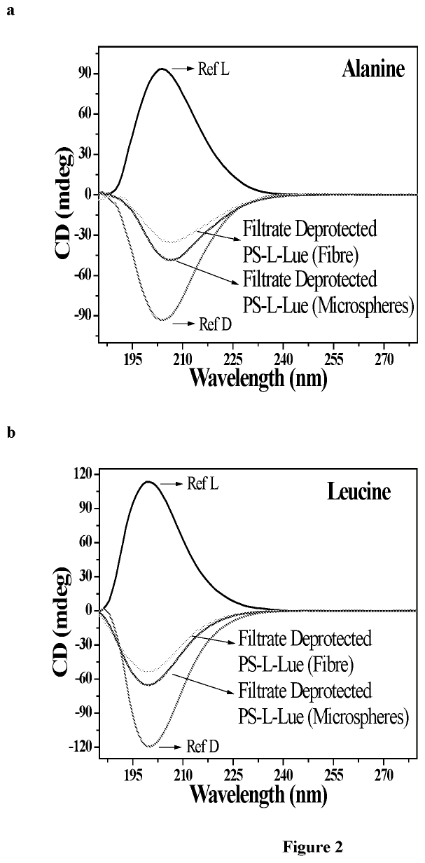

The CD spectrum (FIG. 1 and FIG. 2) of these filtrate solutions is then plotted against corresponding amino acid L and D enantiomers. The percentage enantiomeric excess (% ee) for separation is calculated by taking the ratio of area under the curve for the filtrate with that of the area under curve for the reference D enantiomer solution. The % ee values are given in Table-1 below.

TABLE 1

| | % Enantiomeric excess values (% ee) | |
|---|---|---|
| Racemic | Protected L-Leu-PS | Deprotected L-Leu-PS |
| mixture | Powder/Microspheres | Fibers/Microspheres |
| Alanine | 16.52/35.24 | 40.56/53.75 |
| Leucine | 19.45/42.06 | 49.51/58.15 |

Significant improvement in % ee values is observed in case of deprotected polymer compared to the protected analogue (powder and microspheres). The enhanced performance of the deprotected polymer could be attributed to the increased hydrophilicity of the polymers due to free amine groups, which significantly improved the surface wettability. Additionally, it is anticipated that the polar amine groups improved the enantioselective separation ability by self-assembling in fibrous morphology with the deprotected L-leucine exposed to the polymer surface enhancing the surface chirality.

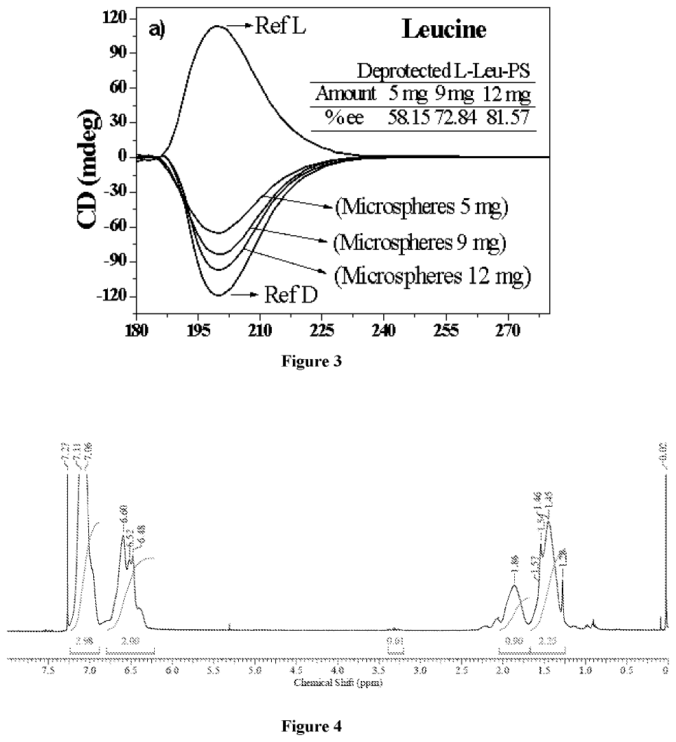

By assembling into microspheres, the overall surface area is enhanced compared to the fibrous morphology. The improvement in the surface chirality reduces the non-enantioselective adsorptions on the surface, resulting in % ee improvement. In case of deprotected polymer, better enantioselective separation is achieved in case of microspheres compared to fibers due to the enhanced surface area. Increasing the amount of deprotected polymer microspheres used for the separation resulted in increased ee % (FIG. 3). For instance, increasing the amount of deprotected PS-L-Leu microspheres from 5 mg to 9 mg for the enantioselective separation of a racemic mixture of leucine, resulted in enhancement of ee % from 58.15 to 72.84, which is further increased to 81.57% upon using 12 mg of polymer microspheres.

FIG. 10 depicts the GPC chromatogram of polymers, which shows that the polystyrene having number average molecular weight 22800 Daltons and weight average molecular weight of 30100 Daltons with the narrow polydispersity of (1.32) is synthesized by RAFT polymerization method. Upon modification with phthaloyl protected L-leucine average molecular weights of the polystyrene slightly altered (Mn 18500, Mw 32300 with PDI of 1.7) and (Mn 16700, Mw 29400 with PDI of 1.8) for protected and deprotected polymers respectively. This change in molecular weight values is attributed to the change in hydrodynamic volumes of the polymers upon modification and deprotection.

Table-2 below summarizes the data related to average molecular weights (Mn and Mw), polydispersity index (Ð_{M}), and yield of polymer and modified polymer.

TABLE 2

| Polymers | Mn | Mw | Ð_{M} | % Yield |
|---|---|---|---|---|
| Polystyrene | 22800 | 30100 | 1.32 | 65 |
| Protected PS-L-Leu (III) | 18500 | 32300 | 1.7 | 68 |
| Deprotected PS-L-Leu (I) | 16700 | 29400 | 1.8 | 74 |

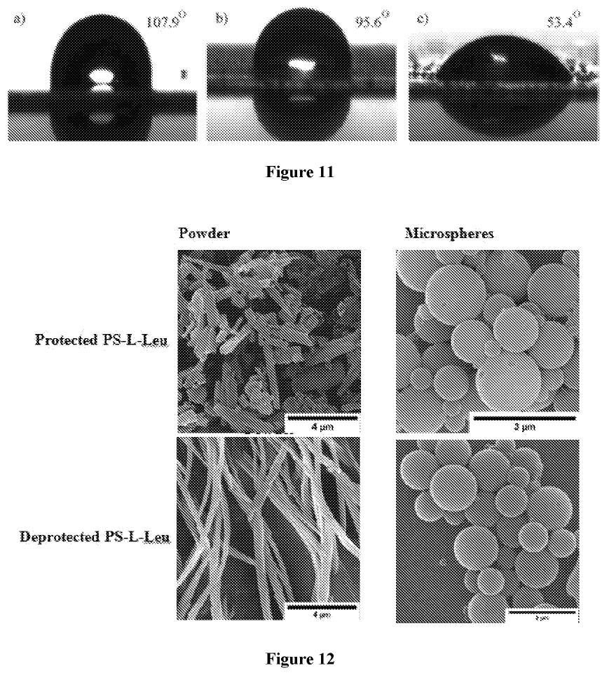

FIG. 11 depicts water contact angles of a) Polystyrene b) Protected PS-L-Leu (III) c) Deprotected PS-L-Leu (I). It shows that a deprotected polymer is hydrophilic as compared to the protected polymer. FIG. 12 depicts the polymer morphology of protected PS-L-Leu (III), and deprotected PS-L-Leu (I) polymers as it is and its microspheres. It shows that protected PS-L-Leu (III) show irregular morphology, upon deprotection this irregular morphology changes to fibrous. This change in morphology from irregular to fibrous is attributed to change in polarity due to free amine. These polar free amines projected themselves towards the polymer surfaces resulting in fibrous morphology.

### Detection and Machines Used:

1H NMR spectra were recorded on Bruker Avance 200 and 400 MHz spectrometers in CDCl3. Chemical shifts (8) are reported in ppm at 25° C. using CDCl3 as the solvent containing a trace amount of tetramethylsilane as an internal standard.

Molecular weights of polymers were measured on a ThermoFinnigan-make gel permeation chromatograph, with a refractive index detector at room temperature. HPLC-grade chloroform was used as an eluent with a flow rate maintained at 1 mL/min.

The gel permeation chromatography (GPC) column was standardized using PS standards with narrow polydispersity. The samples were prepared by dissolving a 3 mg of polymer in 1.5 mL of chloroform and filtered through a 0.45 μm PTFE filter.

Infrared (IR) spectra were recorded on a Bruker α-T spectrophotometer. Sample pellets were prepared by mixing with 5% w/w in KBr and dried under vacuum before the spectra were recorded.

The polymeric microspheres were prepared by using a polymeric solution in DCM and a 4% Tween 80 surfactant solution. The polymer and surfactant solutions were mixed in a 1:4 v/v ratio and homogenized using the RT Miccra D-9 Digitronic Homogenizer Disperser on Stand at 16,000-21,000 rpm for 15 min. The polymeric microsphere suspension was centrifuged on a Remi PR-24 research compufuge centrifuge with a 6×15 mL rotor head at 10,000 rpm.

The average particle size (Z-average) and polydispersity index (PDI) of aqueous dispersion of polymeric microspheres were measured using a Zetasizer ZS 90 apparatus from Malvern Instruments at a fixed angle of 90° at 25° C.

The zeta potential (C) of polymeric microspheres was measured at pH 7 using 0.1 mg/mL dispersion in 0.1 mM KCl.

The morphological characterizations of polymeric samples and microspheres were performed using field emission scanning electron microscopy (FE-SEM) with an FEI Nova Nano SEM 450.

The polymer solution in THF (0.2 mg/mL) was drop cast on precleaned silicon wafers.

In the case of polymer microspheres, the aqueous dispersion was drop cast on precleaned silicon wafers. The samples were dried overnight under reduced pressure at 45° C. The sample drop cast on silicon wafers were directly mounted on the top of the grooved edge of the aluminum SEM specimen stub with a carbon tape.

Before conducting morphological studies, the samples were coated with a 5 nm thick gold film by the sputtering method. The water contact angle of polymers was recorded using the Drop Shape Analyzer-DSA25-KRUSS GmbH.

Solution-state circular dichroism (CD) measurements were made using a JASCO-815 CD spectrometer equipped with a Jasco PTC-424 S/15 Peltier system. 10 mm path length quartz cuvettes were used for a sample volume of 3 mL in Milli-Q water or THF at 25° C. Three scans were averaged for each sample, with a scanning rate of 100 nm/min.

### EXAMPLES

Following examples are given by way of illustration and therefore should not be construed to limit the scope of the invention.

### Example 1

### General Process for the Enantioselective Separation of Amino Acids

Racemic mixtures of native amino acids like alanine, and leucine, were prepared by dissolving 10 mg of each D and L enantiomers in 10 ml of DI water. 5 mg of Protected/deprotected PS-L-Leu powder was added to each vial and the racemic mixtures were stirred along with the polymer suspension on a magnetic stirrer for 24 hours at 25-30° C. After 24 h, the mixtures from the vials were filtered with the help of Whatman filter paper to remove the solid polymeric particles. The CD spectra of the filtrates were recorded for each sample. Similar enantioselective separation experiments were set up by using microspheres Protected/deprotected PS-L-Leu polymers.

### Example 2

### Synthesis of Polystyrene (II) by RAFT Polymerization

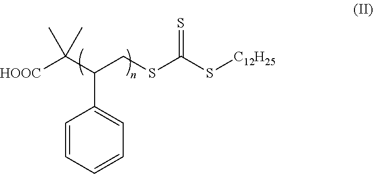

AIBN 15 mg (0.0907 mmols) and CTA (chain transfer (RAFT) agent) 52 mg (0.1439 mmols) taken in a Schlenk tube equipped with a magnetic stirring bar. Distilled styrene monomer 4.5 gm (43.206) was added to the reaction mixture with the help of a syringe through a rubber septum. The reaction mixture was then degassed by three freeze pump thaw cycles. The tube was charged with N₂ gas to maintain the inert atmosphere. The reaction mixture was then heated at 110-115° C. The reaction is allowed to stir at the same temperature for 5-6 hours (until the reaction mixture becomes so viscous that the magnetic bead stops stirring). The Schlenk tube was then cooled in an ice bath which solidified the formed polymer completely, followed by dissolving a formed polymer in a minimum quantity of THF. The formed polymer was precipitated in excess of MeOH. The precipitation was repeated three times to ensure the complete removal of oligomers if formed during polymerization. Yield: 65%, 1H NMR spectrum (400 MHZ instrument, CDCl₃) δ 7.11, 7.06, (m 3H), δ 6.60, 6.52, 6.48 (M 2H), δ 1.86 (s 1H), δ 1.57-1.45 (s 2H). ¹³C NMR spectrum (400 MHZ instrument, CDCl₃) δ 145.29, δ 127.93, 127.63, 127.40, δ 125.62, δ 125.46, δ 43.76, δ 40.35, FT-IR (CHCl₃) stretching frequency (v) in cm⁻¹:3065, 3025, 2926, 2845, 1599, 1497, 1445. GPC (CHCl₃): Number average molecular weight (Mn) 22800 corresponding to the degree of polymerization (Xn=218).

### Example 3

### Synthesis of Protected PS-L-Leu Polymer (III)

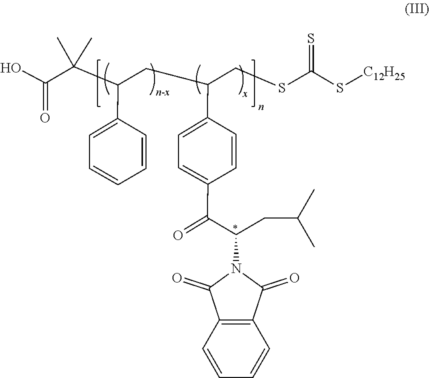

N-phthaloyl 1-leucine acid chloride (1.6 g, 5.7 mmols) was dissolved in 30 mL 1, 2-Dichloroethane in a 100 ml round bottom flask. The reaction mixture was cooled to 0° C. in ice bath. The polystyrene (0.6 g, 5.7 mmols) powder was gradually added to the above solution at 0° C. with continuous stirring. After the addition, the reaction mixture was allowed to stir for 30 minutes at the same temperature. The AlCl₃ (3 g, 22.8 mmols) was added portion-wise to the reaction mixture. The reaction mixture was then allowed to stir at 0° C. for 1 hour.

The ice bath was removed, and the temperature of the reaction mixture was raised to 25-30° C. The reaction mixture was allowed to continue for 2 hours with continuous stirring. The reaction mixture was filtered through Whatman filter paper. The solvent evaporated at reduced pressure on the rotary evaporator. The product was washed with 10% aqueous hydrochloric acid solution (HCl) followed by 10% aqueous sodium hydroxide solution (NaOH) and distilled water and finally with ethanol to ensure the complete removal of unreacted N-phthaloyl 1-leucine acid chloride. The product obtained was dried in an oven at 80° C. for 12 hours.

Yield 68%. 1H NMR spectrum (200 MHZ instrument, CDCl₃) δ 7.84-7.73, (b 2H), δ 7.75-7.73, (m 2H), δ 5.02, 5.0, 4.96, 4.94 (d 1H), δ 2.33-2.01 (d 1H), δ 1.99-1.94 (m 1H). δ1.50 (m 1H) δ 0.96-0.90 (s 6H) FT-IR stretching frequency (υ) in cm⁻¹:3005, 2926, 2863, 1709, 1590, 1445. 1382.

### Example 4

### Synthesis of Deprotected PS-L-Leu (I)

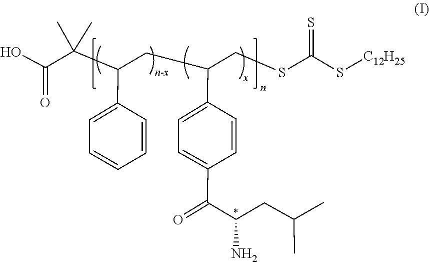

500 mg of N-Phthaloyl L-leucine Containing Polystyrene (N-Pth-L-Leu-PS) powder was dissolved in 50 ml toluene in 2 neck 100 ml round bottom flask. 10 ml of hydrazine hydrate was added to the reaction mixture. Further, the reaction mixture was refluxed at 120° C. for 24 hours. After the completion of the reaction, the mixture cooled to 27° C. The toluene was evaporated at reduced pressure on a rotary evaporator. The reaction mixture was dissolved in DCM.

The organic layer was washed with 2% aqueous KOH solution followed by washing with brine solution. The organic layer dried over anhydrous sodium sulphate (Na₂SO₄). The deprotected polymer was dried completely by solvent evaporation on a rotary evaporator. Complete solvent evaporation resulted in the off-white-coloured polymer.

Yield 74%. 1H NMR spectrum (400 MHZ instrument, CDCl₃) δ 7.01 (b) 6.48 (b) δ 4.92, (b) δ 3.63, (b), δ 1.69 (b) δ 1.26 (b), δ 0.89. FT-IR stretching frequency (v) in cm⁻¹:3035, 2961, 2868, 1771, 1713, 1628, 1466, 1388.

### Example 5

### Preparation of Polymeric Microspheres

150 mg of modified polystyrene powder (Protected PS-L-Leu (III) or Deprotected PS-L-Leu (I)) dissolved in 10 ml of HPLC grade dichloromethane. Separately 4% w/v tween 80 (polyoxyethylene sorbitan monooleate) surfactant prepared by dissolving 4 gram of tween 80 and making up the volume with water in 100 ml volumetric flask. 40 ml of the tween 80 surfactant solution was added to the 10 ml of each modified polystyrene solutions.

The solution was homogenized with the help of a homogenizer at 20000-22000 rpm for 10-15 min until the solution formed a milky white uniform suspension. Homogenization results in the formation of polymeric droplets of uniform size. The shape and size of the polymeric droplet were stabilized by surfactant.

Further, the dichloromethane was evaporated by subjecting the homogenized polymeric suspension to continuous stirring at 1500 rpm with an overhead stirrer for 3-4 h at 45° C. Prolong stirring at 45° C. results in the complete evaporation of dichloromethane from the suspension. The suspension of spherical polymeric particles was then centrifuged at 10000 rpm. As the polymeric particles settled down at the bottom, the supernatant was decanted.

The settle-down polymer particle was washed multiple times (3 times) by adding fresh DI water to ensure the complete removal of the surfactant. Microsphere suspension was then filtered through 0.22 μm pore size PVDF membranes using vacuum filtration assembly. The polymeric particles on the PVDF membrane were then dried overnight in a vacuum oven at 45° C. This results in the formation of spherical polymeric particles from the amino acid modified polystyrenes Protected PS-L-Leu (III) and Deprotected PS-L-Leu (I) polymers.

### Advantages of the Invention

The present invention provides enantioselective separation process by using cheap commodity polymer (polystyrene) after simple post polymer modification

The present invention provides the enantioselective separation process that does not used any high-end chromatographic instruments

The process of present invention separates the amino acid racemic in its native form directly from its aqueous solution without any modification.

The process of present invention achieves high % ee.

The process of present invention achieves separation efficiency of up to 82% within 24 h.

The process of present invention is easy to scale up the chiral microspheres.

1-11. (canceled)

12. A chiral modified polystyrene in the form of a powder, a fiber, or a microsphere, the chiral modified polystyrene being represented by compounds of formula (I): 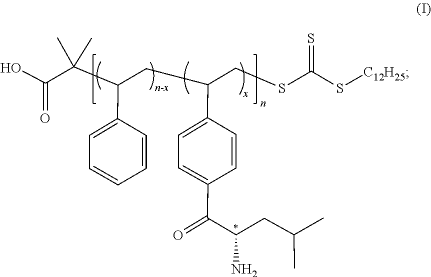
where:
n is from 200 to 250;
x is from 68 to 85; and
n−x is from 132 to 165; and
or by compounds of formula (III): 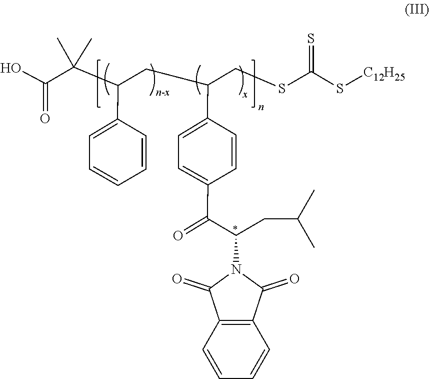
where:
n is from 200 to 250;
x is from 68 to 85; and
n−x is from 132 to 165.

13. The chiral modified polystyrene of claim 12, wherein the chiral modified polystyrene is a compound of formula (I) having a water contact angle of 53.4° or a compound of formula (III) having a water contact angle of 95.6°.

14. A process for preparing the chiral modified polystyrene of claim 12, the process comprising:
(a) synthesizing a polystyrene of formula (II) by control radical polymerization techniques of RAFT polymerization at a temperature from 110° C. to 115° C. for 5 hours to 6 hours as a bulk polymerization without any solvent, according to the following reaction: 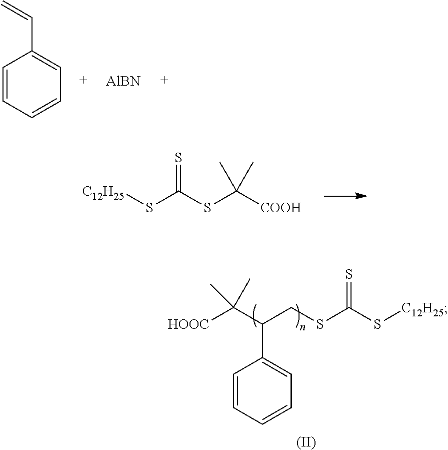
(b) reacting the polystyrene of formula (II) obtained in (a) with N-phthaloyl L-leucine acid chloride in the presence of AlCl₃ at a temperature from 0° C. to 5° C. for 1 hour in a solvent followed by stirring at 25° C. to 35° C. for 2 hours to obtain protected N-phthaloyl L-leucine containing polystyrene (N-Pth-L-Leu-PS) of formula (III), according to the following reaction: 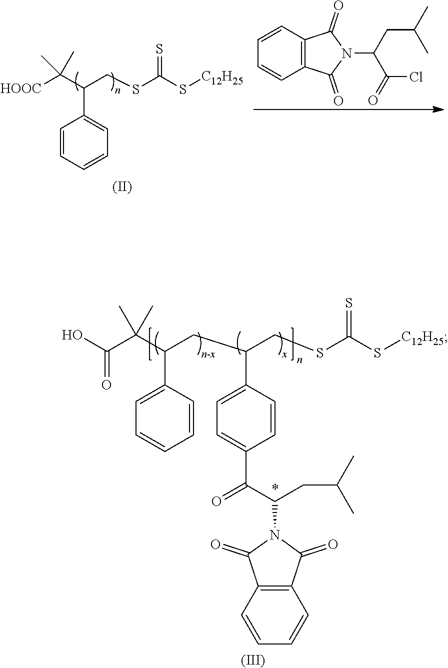
and
(c) deprotecting phthaloyl from the compound of formula (III) obtained (b) in the presence of hydrazine hydrate at a temperature from 120° C. to 125° C. for 22 hours to 24 hours in a solvent to obtain a L-leucine containing polystyrene (PS-L-Leu) of formula (I), according to the following reaction:

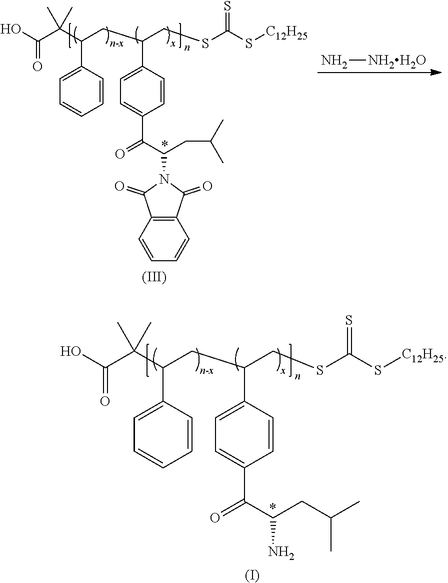

15. The process of claim 14, wherein the yield of the compound of formula (III) in (b) is 68%.

16. The process of claim 14, wherein the yield of the compounds of formula (I) in (c) is 74%.

17. A process for preparing microspheres of the chiral modified polystyrene of claim 12, the process comprising:
(a) dissolving a powder of the chiral modified polystyrene in dichloromethane to obtain a modified polystyrene solution, wherein the powder of the chiral modified polystyrene powder is a protected polystyrene of formula (III) or a deprotected polystyrene of formula (I);
(b) adding 4% aqueous polyoxyethylene sorbitan monooleate surfactant into the modified polystyrene solution obtained in (a) to obtain a reaction mixture, and homogenizing the reaction mixture at 20000 rpm to 22000 rpm with a homogenizer for 10 minutes to 15 minutes until the reaction mixture forms a milky white uniform suspension;
(c) evaporating the dichloromethane from the milky white uniform suspension obtained (b) by subjecting to stirring at 1500 rpm with an overhead stirrer for a 3 hours to 4 hours at a temperature from 40° C. to 45° C. to obtain a polymer particle suspension mass;
(d) centrifuging the polymer particle suspension mass obtained in (c) at 10000 rpm to settle down the polymeric particles at the bottom and then decanting the supernatant liquid to obtain settle-down polymer particles;
(e) washing the settle-down polymer particles obtained in (d) with water and filtering through 0.22 μm pore size PVDF membranes using a vacuum filtration assembly to obtain particles; and
(f) drying the particles obtained in (e) in a vacuum oven at a temperature from 40° C. to 45° C. for 15 hours to 20 hours to afford spherical microspheres polymeric particles.

18. The process of claim 17, wherein the modified polystyrene powder is a deprotected polystyrene of formula (I).

19. The process of claim 17, wherein the deprotected polystyrene microspheres have an enantiomeric excess values (% ee) of 58.15.

20. The process of claim 17, wherein, in (b), the modified polystyrene powder solution and polyoxyethylene sorbitan monooleate surfactant solution are present in a ratio from 1:3 to 1:5.

21. A process for enantioselective separation of a racemic mixture of native amino acid using the chiral modified polystyrene of claim 12, the process comprising:
(a) dissolving a racemic mixture of native amino acid in water under stirring;
(b) adding the chiral modified polystyrene into the racemic mixture obtained of (a) to obtain a polymer powder suspended racemic mixture;
(c) stirring the polymer powder suspended racemic mixture of (b) at a temperature from 25° C. to 35° C. for a period 22 hours to 24 hours to obtain a mixture; and
(d) filtering the suspended polymer powder from the mixture obtained in (c) to afford enantioselective separation, wherein one enantiomer is in a filtrate and another enantiomer is adsorbed on a chiral surface of the polymer powder;
wherein:
the chiral modified polystyrene of (b) is L-leucine containing polystyrene (PS-L-Leu), which is either protected or deprotected; and
the chiral modified polystyrene is in the form of a powder, a fiber or a microsphere.

22. The process of claim 21, wherein the native amino acid is selected from alanine, leucine, aspartic acid, glutamic acid, lysine, phenylalanine, serine, or valine.
